(19) United States
(12) Patent Application Publication
(10) Pub. No.: US 2019/0365657 A1
(43) Pub. Date: Dec. 5, 2019

(54) TUMOR-TARGETING BEAD VECTORS AND METHODS OF USING THE SAME

(71) Applicant: ORBIS HEALTH SOLUTIONS LLC, Greenville, SC (US)

(72) Inventor: Thomas E. Wagner, Greenvillie, SC (US)

(73) Assignee: ORBIS HEALTH SOLUTIONS, LLC, Greenville, SC (US)

(21) Appl. No.: 16/325,859

(22) PCT Filed: Aug. 17, 2017
(86) PCT No.: PCT/US2017/047308

§ 371 Date: Feb. 15, 2019

Related U.S. Application Data
(60) Provisional application No. 62/376,033, filed on Aug. 17, 2016.

(51) Int. Cl. A61K 9/50; A61K 38/16; A61K 35/761; A61K 47/69; C07K 14/705; C07K 16/28; C12N 15/86

(52) U.S. Cl. CPC A61K 9/5094; A61K 38/162; A61K 35/761; A61K 47/6901; A61K 9/0019; A61K 47/6927; C07K 14/70503; C07K 16/2818; C12N 15/86; A61K 47/6923

(57) ABSTRACT

The disclosed bead vectors direct entry into a cell of monocytic origin and cause the expression of an extracellular domain of PD-1, an anti-CTLA4 antibody, or an antibody that is specific for a checkpoint protein. The bead vectors can comprise a nucleic acid component, a lysosome evading component and a bead particle that can be phagocytized. The disclosed vectors are useful in various methods of cancer therapy, treatment, and prevention. Due to the ability of monocytic cells to target tumors, the disclosed vectors are particularly well suited for use in anti-tumor applications and directing expression of target genes in tumor-associated macrophages.

### CROSS-REFERENCE TO RELATED APPLICATION

This application is a National Phase entry of International Application No. PCT/US2017/047308, filed on Aug. 17, 2017, which claims the benefit of and priority to U.S. Provisional Appl. No. 62/376,033, filed Aug. 17, 2016, the contents of which are herein incorporated by reference in their entirety.

### FIELD OF INVENTION

The present disclosure relates generally to the field of cancer therapy, and, in particular, targeted cancer therapy using monocyte-specific bead vectors. The disclosure provides compositions and methods for effectively treating tumors by activating tumor-associated macrophages to express an extracellular domain of programmed death 1 (PD-1) or an anti-checkpoint protein antibody or binding fragment thereof, such as a single-domain antibody that binds to cytotoxic T-lymphocyte-associated protein 4 (CTLA4).

### BACKGROUND OF THE INVENTION

The following discussion is merely provided to aid the reader in understanding the disclosure and is not admitted to describe or constitute prior art thereto.

Monocytic cells normally patrol the body in search of foreign, non-self antigens, such as bacteria. Monocytic cells phagocytize the bacteria, which are then digested to smaller antigenic portions in the lysosome. The resultant bacterial antigens are cycled back to the cell surface of these cells for presentation to the humoral and cellular arms of the immune system.

Monocytes can differentiate into macrophages or dendritic cells after migrating from the blood stream into particular tissues. Importantly, many solid tumors have a vast presence of macrophage cells within the tumor bed. These tumor-associated macrophages (TAMs) are attracted to the hypoxic and/or necrotic microenvironments of the tumor, where they can serve to promote tumor growth and progression through various pathways, such as activation of nuclear factor-kappa B (NF-κB) and the release of pro-angiogenic signals (e.g., VEGF).

Thus, it would be beneficial to utilize TAMs in such a way that would reverse their innate protumoral activity by causing them to express therapeutic proteins instead.

### SUMMARY OF THE INVENTION

Described herein are compositions and methods for treating tumors using monocyte-specific bead vectors for directing expression of therapeutic proteins.

In one aspect, the present disclosure provides bead vectors comprising (i) a nucleic acid encoding a PD-1 extracellular domain or a single-domain antibody that binds to cytotoxic T-lymphocyte-associated protein 4 (CTLA4), (ii) a lysosome evading component, and (iii) a bead particle that can be phagocytized.

In another aspect, the disclosure provides bead vectors for directed entry into a monocyte cell comprising (i) a nucleic acid encoding a PD-1 extracellular domain or a single-domain antibody that binds to CTLA4, (ii) a lysosome evading component, and (iii) a bead particle that is about 0.5 to about 2.5 microns and allows the composition to be phagocytized by a monocytic cell.

In another aspect, the disclosure provides methods of treating cancer in a patient comprising administering to a patient with cancer a bead vector comprising (i) a nucleic acid encoding a PD-1 extracellular domain or a single-domain antibody that binds to CTLA4, (ii) a lysosome evading component, and (iii) a bead particle that is about 0.5 to about 2.5 microns, such as a yeast cell wall particle, wherein administration of the bead vector treats the patient's cancer.

In another aspect, the present disclosure provides bead vectors comprising (i) a nucleic acid encoding an anti-checkpoint protein antibody or binding fragment thereof, (ii) a lysosome evading component, and (iii) a bead particle that can be phagocytized.

In another aspect, the disclosure provides bead vectors for directed entry into a monocyte cell comprising (i) a nucleic acid encoding an anti-checkpoint protein antibody or binding fragment thereof, (ii) a lysosome evading component, and (iii) a bead particle that is about 0.5 to about 2.5 microns and allows the composition to be phagocytized by a monocytic cell.

In another aspect, the disclosure provides methods of treating cancer in a patient comprising administering to a patient with cancer a bead vector comprising (i) a nucleic acid encoding an anti-checkpoint protein antibody or binding fragment thereof, (ii) a lysosome evading component, and (iii) a bead particle that is about 0.5 to about 2.5 microns, such as a yeast cell wall particle, wherein administration of the bead vector treats the patient's cancer.

In some embodiments, the lysosome evading component may be a non-infectious virus or non-infectious component of a virus. For example, the non-infectious virus can be an adenovirus or a recombinant adenovirus. Additionally, in some embodiments, the non-infectious virus or non-infectious component of a virus can be non-replicative.

In some embodiments, the nucleic acid encoding a PD-1 extracellular domain or a single-domain antibody that binds to CTLA4 can be selected from the group consisting of DNA and RNA. In some aspects, the anti-checkpoint protein antibody or binding fragment thereof is an anti-PD-L1 antibody or binding fragment thereof, while in some embodiments, the anti-checkpoint protein antibody is an anti-CTLA4 antibody or binding fragment thereof. In some embodiments, the anti-checkpoint antibody may be a single-domain antibody.

In some embodiments, the PD-1 extracellular domain comprises a mammalian PD-1 extracellular domain, such as human or murine PD-1 extracellular domain.

In some embodiments, the nucleic acid encoding a PD-1 extracellular domain, a single-domain antibody that binds to CTLA4, or the anti-checkpoint protein antibody or binding fragment thereof can be encoded in an expression vector. In some embodiments, the expression vector can comprise a nuclear promoter, such a CMV promoter. In some embodiments, the expression vector can comprise a hypoxia-induced promoter, such as a chimeric promoter of HREx3+Basal SV40 promoter. In some embodiments, the expression vector can comprise a T7 promoter, FSV non-structural protein genes, and/or a FSV subgenomic promoter.

In some embodiments, the bead vectors can comprise a nucleic acid protecting component, such as protamine, polyarginine, polylysine, histone, histone-like proteins, synthetic polycationic polymers, or a core particle of a virus with the appropriate packaging sequence included in the nucleic acid sequence (e.g., the DNA or RNA sequence).

In some embodiments, the nucleic acid encoding a PD-1 extracellular domain, a single-domain antibody that binds to CTLA4, or the anti-checkpoint protein antibody or binding fragment thereof and the lysosome evading component can be attached to the bead particle by interaction between streptavidin and biotin. In some embodiments, the nucleic acid encoding a PD-1 extracellular domain, a single-domain antibody that binds to CTLA4, or the anti-checkpoint protein antibody or binding fragment thereof and the lysosome evading component can be attached to the bead particle by antibody attachment.

In some embodiments, the bead particle can comprise a ferro-magnetic particle, a microbead, a microsphere, or a yeast cell wall particle (YCWP).

In some embodiments, the monocytic cell that phagocytizes the bead vector is a macrophage, such as a tumor-associated macrophage (TAM).

In some embodiments, the cancer being treated expresses a PD-1 ligand or CTLA4. And in some embodiments, the cancer comprises at least one tumor that may have a hypoxic microenvironment and may further comprise tumor associated macrophages (TAMs).

In some embodiments, the disclosed bead vector is administered intradermally or subcutaneously. For instance, in some embodiments, the disclosed bead vector may be administered intradermally or subcutaneously in proximately to a target lymph node (i.e., the lymph node that is closest to the tumor being treated).

The foregoing general description and following detailed description are exemplary and explanatory and not limiting of the disclosure.

### BRIEF DESCRIPTION OF THE DRAWINGS

FIG. 1 shows the full length amino acid sequence of human (A) PD-1 and (B) murine PD-1. The extracellular domains of each protein are indicated in bold.

FIG. 2 shows an exemplary viral entry vector for expressing a mouse PD-1 ectodomain.

FIG. 3 shows an exemplary step-wise scheme for attaching a recombinant viral vector to a bead particle to form an exemplary bead vector delivery system.

FIG. 4 shows an exemplary step-wise scheme for producing recombinant viral particles for attachment to the disclosed bead particles.

FIG. 5 shows tumor volume of mice with B16 murine melanoma xenografts following intratumoral injection of control particles (for expressing GFP) and particles for expressing a PD-1 extracellular domain.

FIG. 6 shows exemplary mice with B16 melanoma xenografts four weeks after a single intratumor injection of either control particles (for expressing GFP) and particles for expressing a PD-1 extracellular domain. The mouse in panel (A) is a control mouse, while the mouse in panel (B) received bead particles for expressing a PD-1 extracellular domain.

FIG. 7 shows an exemplary vector for expressing an anti-CTLA4 single-domain antibody.

FIG. 8 shows the full length sequence of an exemplary vector for expressing an anti-CTLA4 single-domain antibody.

### DETAILED DESCRIPTION

In general, the present disclosure provides novel, targeted gene delivery vectors and methods of using the same. In particular, the disclosure provides bead- or yeast cell wall particle (YCWP)-based delivery vectors for expressing a single-domain antibody that binds to CTLA4 or for expressing the ectodomain or extracellular domain of PD-1 in tumor-associated macrophage (TAM) cells and the microenvironment of the tumor, and the expression of the antibody or the PD-1 ectodomain or extracellular domain may be induced by hypoxia in the tumor microenvironment.

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

### Definitions

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the phrases “therapeutically effective amount” means that a dose of the disclosed bead particles provides the specific pharmacological effect for which the drug is administered in a subject in need of such treatment, i.e. to reduce, ameliorate, or eliminate cancer/tumor growth, progression, or recurrence. It is emphasized that a therapeutically effective amount of a bead particle will not always be effective in treating the cancer/tumors of every individual subject, even though such dosage is deemed to be a therapeutically effective amount by those of skill in the art. Those skilled in the art can adjust what is deemed to be a therapeutically effective amount in accordance with standard practices as needed to treat a specific subject and/or specific type of cancer or tumor. The therapeutically effective amount may vary based on the route of administration and dosage form, the age and weight of the subject, and/or the subject's condition, including the progression, stage, and/or class of cancer or tumor at the time of treatment.

The terms “treatment” or “treating” as used herein with reference to cancer or tumors refer to reducing, ameliorating or eliminating cancer/tumor growth and/or progression, or causing caner/tumor cell death.

The terms “prevent” or “preventing” as used herein refer to stopping the formation of cancer/tumor cells or inhibiting the recurrence of cancer/tumor growth.

The terms “individual,” “subject,” and “patient” are used interchangeably herein, and refer to any individual mammalian subject, e.g., bovine, canine, feline, equine, or human.

The compositions and methods of the disclosure may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

### Programmed Death-1 (PD-1)

PD-1 is a 50-55 kDa type I transmembrane receptor that was originally identified in a T cell line undergoing activation-induced apoptosis. PD-1 is expressed on T cells, B cells, and macrophages. The ligands for PD-1 are the B7 family members PD-L1 (B7-H1) and PD-L2 (B7-DC).

PD-1 is a member of the immunoglobulin (Ig) superfamily that contains a single Ig V-like domain in its extracellular region. The PD-1 cytoplasmic domain contains two tyrosines, with the most membrane-proximal tyrosine (VAYEEL in mouse PD-1) located within an ITIM (immuno-receptor tyrosine-based inhibitory motif). The presence of an ITIM on PD-1 indicates that this molecule functions to attenuate antigen receptor signaling by recruitment of cytoplasmic phosphatases.

Experimental data implicates the interactions of PD-1 with its ligands in the downregulation of central and peripheral immune responses.

The present disclosure provides compositions and methods for activating the immune system by engineering tumor-associated macrophages (TAMs) to produce and secrete the extracellular domain of PD-1. Expression of the PD-1 extracellular domain within a tumor bed can competitively block all of the PD-1 ligands (i.e., PD-L1 and PD-L2) on the tumor surface. In so doing, the disclosed compositions and methods elicit a strong and tumor specific intra-tumor checkpoint inhibition that results in destruction of the tumor and treatment of the disease.

### Cytotoxic T-Lymphocyte-Associated Protein 4 (CTLA4)

CTLA4, also known as CD152 (cluster of differentiation 152), is a protein receptor that, downregulates immune responses by functioning as an immune checkpoint. CTLA4 is constitutively expressed in Tregs but only upregulated in conventional T cells after activation. It acts as an “off” switch when bound to CD80 or CD86 on the surface of antigen-presenting cells. The CTLA4 protein is encoded by the Ctla4 gene in mice and the CTLA4 gene in humans.

CTLA4 is homologous to the T-cell co-stimulatory protein, CD28, and both molecules bind to CD80 and CD86, also called B7-1 and B7-2 respectively, on antigen-presenting cells. CTLA4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA4.

The mechanism by which CTLA4 acts in T cells remains somewhat controversial. Biochemical evidence suggested that CTLA4 recruits a phosphatase to the T cell receptor (TCR), thus attenuating the signal. More recent work has suggested that CTLA4 may function in vivo by capturing and removing B7-1 and B7-2 from the membranes of antigen-presenting cells, thus making these unavailable for triggering of CD28.

Additionally, it has been found, dendritic cell (DC)-Treg interaction causes sequestration of Fascin-1, and skews Fascin-1-dependent actin polarization in antigen presenting DCs toward the T reg cell adhesion zone. Although it is reversible upon T regulatory cell disengagement, this sequestration of essential cytoskeletal components causes a lethargic state of DCs, leading to reduced T cell priming. This suggests Treg-mediated immune suppression is a multi-step process. In addition to CTLA-4 CD80/CD86 interaction, fascin-dependent polarization of cytoskeleton towards DC-Treg immune synapse may play a pivotal role.

The present disclosure provides compositions and methods for activating the immune system by engineering tumor-associated macrophages (TAMs) to produce and secrete an antibody that binds to CTLA4, specifically a single-domain antibody. Expression of the anti-CTLA4 antibody within a tumor bed can competitively block all of the ligands of CTLA4 from binding the receptor on the tumor surface. In so doing, the disclosed compositions and methods elicit a strong and tumor specific intra-tumor checkpoint inhibition that results in destruction of the tumor and treatment of the disease.

### Bead Vectors

The present disclosure provides a solid matrix-based composition for directed entry into a monocyte cell (hereafter a “bead vector”). A basic bead vector according to the present disclosure is generally composed of a nucleic acid component, a lysosome evading component and a bead particle that can be phagocytized by monocytic cells. The bead vector is highly specific for phagocytic cells like monocyte cells, including dendritic cells and macrophages. This high selectivity for monocyte cells renders the bead vector extremely useful for gene therapy and other gene medicine methods, requiring introduction and expression of genes into cells of the monocyte lineage, such as cancer treatment.

The basic structure of the bead vectors used herein is disclosed in U.S. Pat. No. 6,875,612, which is hereby incorporated by reference.

### Bead Particles

The disclosed bead vectors take advantage of the phagocytic activity of monocyte cells by “looking” like a bacterium. Thus, a preferred size for the bead particle is one that approximates the size of the bacterial antigens that monocyte cells typically ingest. Generally, the vector particle will be about 0.5 to about 2.5 microns, or about 0.5 to about 1 micron. Thus, the vector particle may be about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5 microns.

From the perspective of uptake, the smaller end of the ranges is preferred, because it more closely approximate the size of a bacterium. On the other hand, for manufacturing purposes, slightly larger beads are preferred, because they are less likely to stick together, and so washing free from bound components is easier with the larger beads.

Furthermore, the bead particle is not limited by shape or material. The bead particle can be of any shape, size, or material that allows the bead vector to be phagocytized by monocytic cells.

For instance, the bead particle may comprise a ferro-magnetic center covered by a polymer coat. Potentially useful ferro-magnetic particles include, but are not limited to, microbeads, microspheres, and silicate beads. Such beads may be preferred in certain applications because magnetic separation can be employed to separate free from bead-bound components during processing. However, bead particles for use in the disclosed bead vectors are not limited to a specific type of material and may be made of synthetic materials like polystyrene or other plastics, as well as biological materials.

Still other particles that may be suitable as bead particles include yeast cell wall particles (YCWPs), such as yeast glucan particles. YCWPs can be particularly beneficial in the disclosed bead vectors because they may comprise beta glucan, which encourages macrophage phagocytosis of the particles.

A YCWP can be prepared from yeast cell wall such that the particle is porous to the delivery of various macromolecules. In one embodiment, the YCWP can be prepared from Saccharomyces cerevisiae. In another embodiment, the YCWP can a zymosan particle. In another embodiment, the YCWP approximates the size of microbial structures that cells of the mononuclear phagocyte system and other phagocytic cells typically ingests (e.g., bacteria). In specific embodiments, the YCWP can be about 1-5 μm.

In some embodiments, the YCWP may be prepared by (a) suspending yeast to produce a suspension, (b) incubating the suspension, (c) centrifuging the suspension and removing the supernatant and (d) recovering the resulting YCWP. In some embodiments, steps (a)-(d) are repeated at least 1, 2, 3 or 4 times.

In some embodiments, the YCWP may be prepared by (a) suspending yeast in a solution to produce a first suspension, (b) incubating the first suspension, (c) centrifuging the first suspension and removing the supernatant, (d) suspending the resulting pellet to produce a second suspension, (e) incubating the second suspension, (f) centrifuging the second suspension and removing the supernatant and (g) washing the resulting pellet to recover the YCWP. In some embodiments, the YCWP is sterilized.

In some embodiments, the yeast is suspended in NaOH, including 1M NaOH. In some embodiments, the first suspension is incubated at about 80° C. for about 1 hour or for 1 hour. In some embodiments, the centrifuging is performed at about 2000 times gravity for about 10 minutes, or at 2000 times gravity for 10 minutes. In some embodiments, the pellet is suspended in water, including water at about pH 4.5 or at pH 4.5. In some embodiments, the second suspension is incubated at about 55° C. for about 1 hour or at 55° C. for 1 hour. In some embodiments, the pellet is washed in water at least 1, 2, 3 or 4 times. In some embodiments, the pellet is washed once.

In some embodiments, the YCWP is sterilized using isopropanol and/or acetone following washing of the pellet. In specific embodiments, other known alcohols are appropriate. In some embodiments, the YCWP is allowed to fully dry after sterilization. In some embodiments, the YCWP is resuspended after being allowed to dry. In some embodiments, the YCWP is some in PBS, such as 1×PBS. In some embodiment, the YCWP is allowed to dry and then frozen before the tumor lysate is loaded into the YCWP, in order to place it in storage before use. In some embodiments, the YCWP is freeze dried and store at about 4° C. or lower. In some embodiments, the YCWP is freeze dried and store at 4° C.

### Nucleic Acid Component

The basic bead vector of the disclosure may have attached to it a nucleic acid component. The nucleic acid component typically encodes a therapeutic nucleic acid or protein. The nucleic acid component is composed of DNA, RNA or both DNA and RNA. This component typically contains the signals necessary for translation and/or transcription (i.e., it can encode a protein or an RNA product that may be expressed on or secreted by the target cell).

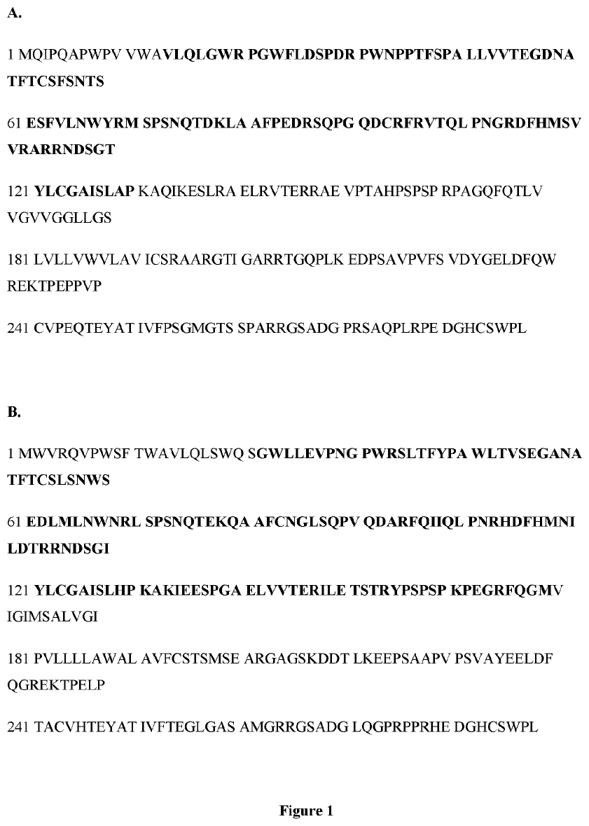

One of skill in the art will comprehend the large number of therapeutic proteins that can be employed in the instant vector system. Typically, they will be anti-tumor proteins. For instance, in some embodiments, the nucleic acid component will encode an extracellular domain of PD-1. The PD-1 extracellular domain may be from human (NP_005009, NM_005018), mouse (NP_032824, NM_008798), bovine (NP_001277851, NM_001290922), or other animal origin. One of skill in the art will be able to identify a suitable extracellular domain of PD-1. For instance, human PD-1 is 288 amino acids in length, and amino acids 14-130 represent the extracellular domain, whereas murine PD-1 is also 288 amino acids but amino acids 21-169 represent the extracellular domain (see FIG. 1).

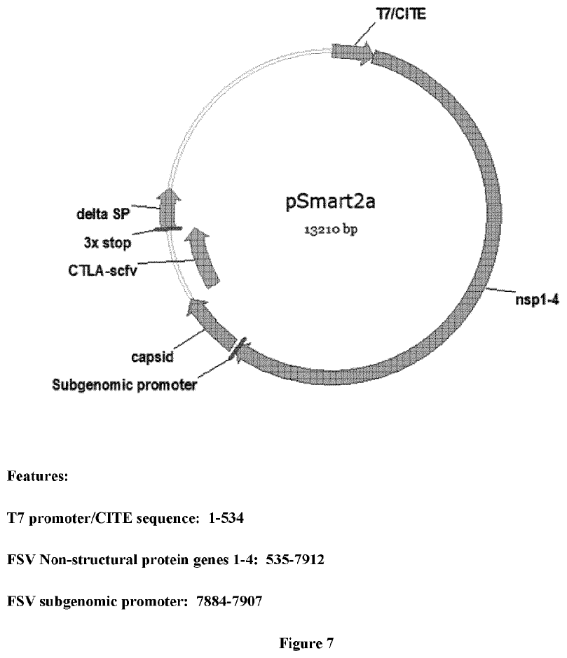

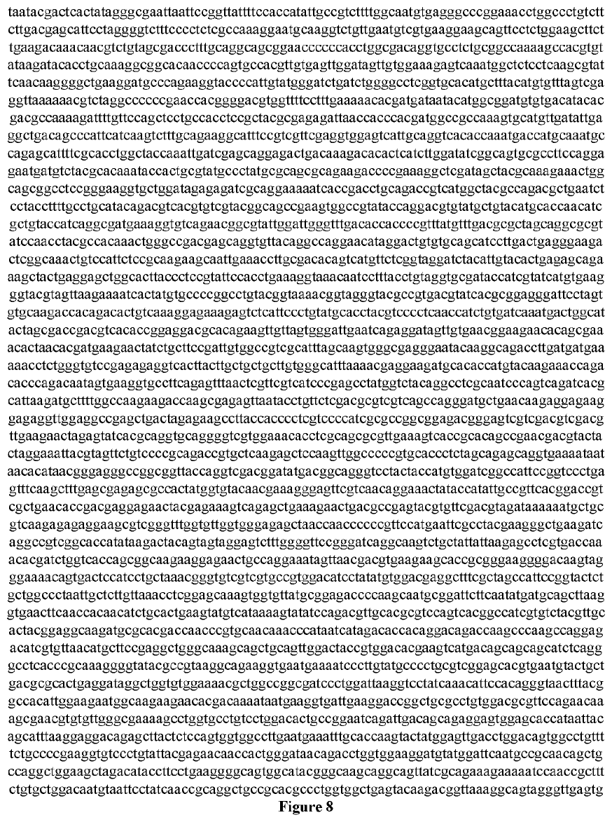

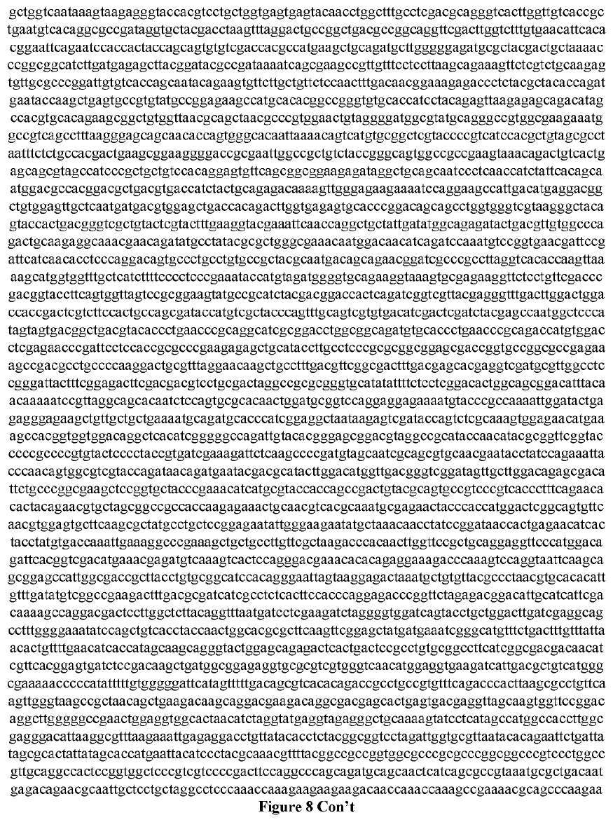

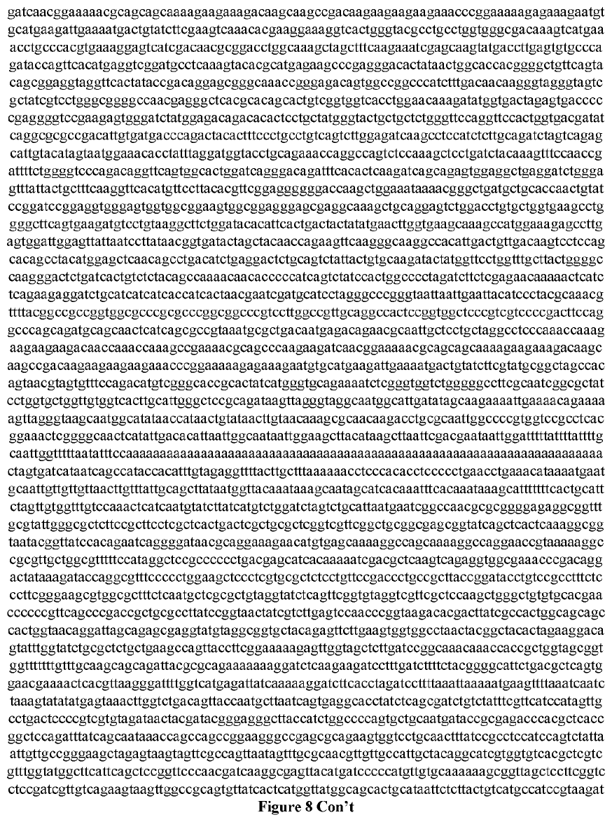

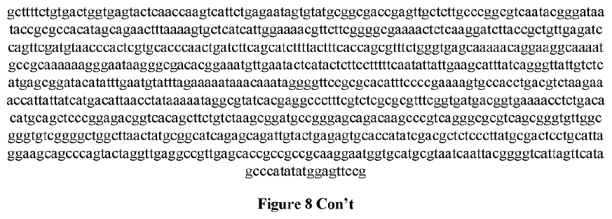

In some embodiments, the nucleic acid component will encode an anti-CTLA4 antibody, in particular, it may encode a single-domain (i.e., short chain) antibody that binds to CTLA4 (as shown in FIG. 7). Numerous anti-CTLA4 antibodies are known in the art, including but not limited to Ipilimumab (YERVOY®) and tremelimumab, and one of skill in the art would readily understand how to express these antibodies or a single-domain antibody comprising the variable regions of these antibodies in the disclosed bead vector. One exemplary anti-CTLA4 antibody is disclosed in US 2011/0044953, which is hereby incorporated by reference, and the sequence of an expression vector for expressing a single-domain version of this antibody is shown in FIG. 8.

Alternatively, in some embodiments, the nucleic acid component may encode an antibody or binding fragment thereof that is specific for an immune checkpoint protein(s). Immune checkpoints are inhibitory pathways, which, under normal conditions are crucial for maintaining self-tolerance and modulating the duration and amplitude of physiological immune responses in peripheral tissues in order to minimize collateral tissue damage in response to pathogenic infection. However, the expression of immune checkpoint proteins is often dysregulated by tumors as an important immune resistance and escape mechanism.

Because many of the immune checkpoints are initiated by ligand-receptor interactions, they can be readily blocked by antibodies or binding fragments specific for the checkpoint ligands and/or receptors. Thus, the nucleic acid component of the disclosed bead vectors may express antibodies or binding fragments thereof that are specific for checkpoint proteins including, but not limited to, those proteins shown in Table 1.

TABLE 1

| Target | Biological Function |
|---|---|
| CTLA4 | Inhibitory Receptor |
| PD1 | Inhibitory Receptor |
| PDL1 | Ligand for PD1 |
| LAG3 | Inhibitory Receptor |
| B7.1 | Costimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Costimulatory Molecule |
| OX-40 | Costimulatory Receptor |
| CD40 | Costimulatory Molecule |
| CD27 | Costimulatory Receptor |
| CCR4 | Costimulatory Receptor |
| GITR | Costimulatory Receptor |
| NKG2D | Activating Receptor |
| KIR | Costimulatory Receptor |

CTLA4, cytotoxic T-lymphocyte-associated antigen 4; LAG3, lymphocyte activation gene 3; PD1, programmed cell death protein 1; PDL, PD1 ligand; TIM3, T cell membrane protein 3; VISTA, V-domain immunoglobulin (Ig)-containing suppressor of T-cell activation; KIR, killer IgG-like receptor.

The antibodies or binding fragments thereof that target the checkpoint proteins are not particularly limited. For instance, the antibodies or binding fragments encoded by the nucleic acid component may be human, chimeric, humanized, or non-human (e.g., mouse, rat, rabbit, sheep, goat, bovine, porcine, etc.). The antibodies may be IgG1, IgG2, IgG3, IgG4, IgA1, IgA2, IgE, or IgM, or variants or fragments thereof.

Anti-checkpoint protein antibody sequences useful in the disclosed bead vector and methods can be obtained by any means, including via in vitro sources (e.g., a hybridoma or a cell line producing an antibody recombinantly) and in vivo sources. Human, partially humanized, fully humanized, and chimeric antibodies can be expressed by the nucleic acid component.

Typically, an antibody consists of four polypeptides: two identical copies of a heavy (H) chain polypeptide and two copies of a light (L) chain polypeptide. Typically, each heavy chain contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody.

However, in some embodiments, the nucleic acid component may encode a binding fragment, rather than an entire antibody. A binding fragment, as used herein, refers to one or more fragments of an anti-checkpoint protein antibody that retain the ability to bind the target protein. Examples of binding fragments include (i) Fab fragments (monovalent fragments consisting of the VL, VH, CL and CH1 domains); (ii) F(ab′)2 fragments (bivalent fragment comprising two Fab fragments linked by a disulfide bridge at the hinge region); (iii) Fd fragments (comprising the VH and CH1 domains); (iv) Fv fragments (comprising the VL and VH domains of a single arm of an antibody), (v) dAb fragments (comprising a VH domain); and (vi) isolated complementarity determining regions (CDR), e.g., VH CDR3. Other examples of binding fragments include single chain Fv (scFv) constructs. See e.g., Bird et al., Science, 242:423-26 (1988); Huston et al., Proc. Natl. Acad. Sci. USA, 85:5879-83 (1988).

In some embodiments, the nucleic acid component may encode a single-domain antibody, which comprises a single monomeric variable antibody domain. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Such “single-domain” antibodies were first engineered from heavy-chain antibodies found in camelids and were are called VHH fragments, but these antibodies may be expressed recombinantly as well.

Thus, in some embodiments, the nucleic acid component of the disclosed bead vectors can comprise a sequence for expressing an anti-PD-L1 antibody or binding fragment thereof, an anti CTLA4 antibody or binding fragment thereof, or another antibody or binding fragment thereof that is specific for any one of the checkpoint proteins disclosed in Table 1.

In some embodiment, the nucleic acid component is encoded in an expression vector, which is capable of expressing the RNA and/or protein products of the nucleic acid component. The encoded protein products may be expressed on or secreted from the target cells. The vector typically further comprises regulatory sequences, including for example, a promoter, operably linked to the coding sequence. The vector may further comprise a selectable marker sequence, for instance for propagation in in vitro bacterial or cell culture systems.

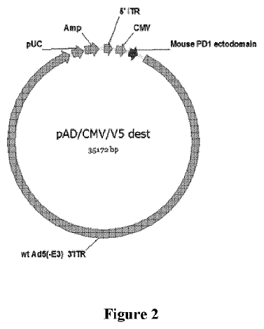

Preferred expression vectors comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 or cytomegalovirus (CMV) viral genome, for example, SV40 origin, early promoter, enhancer, splice, and polyadenylation sites may be used to provide the required non-transcribed genetic elements. Exemplary viral entry vectors are shown in FIGS. 2 and 7. In some embodiments, the promoter may be a T7 promoter.

Specific initiation signals may also be required for efficient translation of inserted target gene coding sequences. These signals can include the ATG initiation codon and adjacent sequences. In some embodiments, a nucleic acid component includes its own initiation codon and adjacent sequences may be inserted into the appropriate expression vector, and no additional translation control signals may be needed. However, in some embodiments, only a portion of an open reading frame (ORF) is used, and exogenous translational control signals, including, for example, the ATG initiation codon, can be provided. Furthermore, the initiation codon may be in phase with the reading frame of the desired coding sequence to ensure translation of the entire target.

These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544 (1987)). Some appropriate expression vectors are described by Sambrook, et al., in Molecular Cloning: A Laboratory Manual, Second Edition, Cold Spring Harbor, N.Y. (1989), the disclosure of which is hereby incorporated by reference. If desired, to enhance expression and facilitate proper protein folding, the codon context and codon pairing of the sequence may be optimized, as explained by Hatfield et al., U.S. Pat. No. 5,082,767.

Promoters include CMV immediate early, HSV thymidine kinase, early and late SV40, LTRs from retrovirus, and mouse metallothionein-I. A preferred promoter is CMV. Exemplary vectors include pWLneo, pSV2cat, pOG44, pXT1, pSG (Stratagene) pSVK3, pBPV, pMSG, and pSVL (Pharmacia). Selectable markers include CAT (chloramphenicol transferase). Preferred vectors also include cytoplasmic vectors, like the T7 vector system. See Wagner et al., U.S. Pat. No. 5,591,601 (Jan. 7, 1997).

In some embodiments, the vector may additionally comprise other functional sequence such as FSV Non-structural protein genes and/or a FSV subgenomic promoter, as shown in FIG. 7.

In some embodiments, the promoter may be inducible. Inducible promoters operably link the expression of target genes (e.g., a PD-1 ectodomain or anti-CTLA4 antibody) to a specific signal or a particular biotic or abiotic factor. Types of inducible promoters that may be utilized in the disclosed expression system include, but are not limited to, chemically-inducible promoters (i.e., antibiotics, steroids, metals, etc.), light-inducible promoters, heat-inducible promoters, and hypoxia-inducible promoters.

In some embodiments, transcription of target genes (e.g., a PD-1 ectodomain or anti-CTLA4 antibody) can be controlled by a hypoxia-inducible promoter. Transcriptional regulation of gene expression under hypoxia can be mediated by hypoxia induced factor 1 (HIF1). The binding of HIF1 to HIF1 responsive elements (HRE) in an enhancer sequence of a promoter leads to gene expression. Several gene promoters have been found to be hypoxia-inducible including, but not limited to, erythropoietin gene, phosphoglycerate kinase-1, and VEGF (The Journal of Experimental Biology 201, 1153-1162, 1998).

Since native promoters are regulated by multiple transcription factors, it is possible to make a chimeric promoter that is more specific to hypoxia (Gene Therapy (2002) 9, 1403-1411). Thus, in some embodiments, a chimeric promoter can be constructed with an enhancerless basal viral promoter, such as SV40 and CMV, and several copies of HRE. For example, in some embodiments, the disclosed expression system can comprise a chimeric promoter of HREx3+Basal SV40 promoter.

Incorporating a hypoxia-inducible promoter into the nucleic acid component of the disclosed bead vectors can increase tumor targeting, as the microenvironment of the tumor is generally the only hypoxic environment in an otherwise healthy body. Thus, if the disclosed bead vectors are administered to a subject systemically and phagocytosed by monocytic cells in circulation, the nucleic acid component will not express the target gene or genes until the monocytic cell has infiltrated into the tumor bed and is exposed to hypoxic conditions. This will result in tumor targeted expression of the nucleic acid component.

### Lysosome Evading Component

In addition to the bead particle and the nucleic acid component, a bead vector of the present disclosure may also comprise a lysosome evading component. The role of the lysosome evading component with respect to the bead vector is to assist the vector in escaping the harsh environment of the lysosome following phagocytosis by a monocyte cell. Aside from those disclosed herein, a person of ordinary skill in the art will be aware of numerous molecules that can serve as a lysosome evading component.

When a monocytic cell ingests a large antigen, a phagocytic vesicle (phagasome) is formed which engulfs the antigen. Next, a specialized lysosome contained in the monocyte cell fuses with the newly formed phagosome. Upon fusion, the phagocytized antigen is exposed to several highly reactive molecules as well as a concentrated mixture of lysosomal hydrolases. These highly reactive molecules and lysosomal hydrolases digest the contents of the phagosome. Therefore, by attaching a lysosome evading component to the particle, the nucleic acid that is also attached to the particle escapes digestion by the materials in the lysosome and enters the cytoplasm of the monocyte intact. Prior systems have failed to recognize the importance of this feature and, thus, obtained much lower levels of expression than the expression systems of the present disclosure. See Falo et al., WO 97/11605 (1997). It should be noted that the term “lysosome evading component” encompasses the fused lysosome/phagosome described above.

The lysosome evading component is any component that is capable of evading or disrupting the lysosome. For example, the lysosome evading component can include proteins, carbohydrates, lipids, fatty acids, biomimetic polymers, microorganisms and combinations thereof. It is noted that the term “protein” encompasses a polymeric molecule comprising any number of amino acids. Therefore, a person of ordinary skill in the art would know that “protein” encompasses a peptide, which is understood generally to be a “short” protein. In some embodiments, lysosome evading components include, but are not limited to, proteins, viruses or parts of viruses.

In some embodiments, the lysosome evading component is a virus that expressed the target gene or genes encoded by the nucleic acid component. The virus may be an RNA virus, like a retrovirus, or a DNA virus, like an adenovirus. In some embodiments, the virus may be recombinant and/or non-replicative and/or non-infective. One of skill in the art will know of commonly used methods to make a virus non-replicative and/or non-infective.

In some embodiments, the lysosome evading component may comprise specific viral proteins. For example, the adenovirus penton protein is a complex that enables a virus to evade/disrupt the lysosome/phagosome. Thus, either the intact adenovirus or the isolated penton protein, or a portion thereof (see, e.g., Bal et al., Eur J Biochem 267:6074-81 (2000)), can be utilized as the lysosome evading component. In some embodiments, fusogenic peptides derived from N-terminal sequences of the influenza virus hemagglutinin subunit HA-2 may also be used as the lysosome evading component (Wagner, et al., Proc. Natl. Acad. Sci. USA, 89:7934-7938, 1992).

Other lysosome evading components include, but are not limited to, biomimetic polymers such as Poly (2-propyl acrylic acid) (PPAAc), which has been shown to enhance cell transfection efficiency due to enhancement of the endosomal release of a conjugate containing a plasmid of interest (see Lackey et al., Abstracts of Scientific Presentations: The Third Annual Meeting of the American Society of Gene Therapy, Abstract No. 33, May 31, 2000-Jun. 4, 2000, Denver, Colo.) Examples of other lysosome evading components envisioned by the present invention are discussed by Stayton, et al. J. Control Release, 1; 65(1-2):203-20, 2000.

In addition to the components described above, a bead vector of the present disclosure may also comprise nucleic acid protection components, either directly or via attachment to one another (e.g., a recombinant adenovirus encoding a nucleic acid component). For example, a DNA protecting component may optionally be added to the basic bead vector described above, especially where the nucleic acid component is not associated with a virus or a portion thereof. Generally, the DNA protecting component will not be attached directly to the bead particle. The nucleic acid protecting component includes any component that can protect bead-bound DNA or RNA from digestion during brief exposure to lytic enzymes prior to or during lysosome disruption. In some embodiments, nucleic acid protecting components include, but are not limited to, protamine, polyarginine, polylysine, histone, histone-like proteins, synthetic polycationic polymers and core protein of a retrovirus with the appropriate packaging sequence included in the nucleic acid sequence (i.e., DNA or RNA sequence).

In some embodiments of the present disclosure, the nucleic acid protection component comprises (1) a recombinant, optionally non-replicative and/or non-infective, form of a virus which contains a nucleic acid encoding an antigen or therapeutic gene that is attached to a bead particle that can be phagocytized. The virus may be an RNA virus, like a retrovirus, or a DNA virus, like an adenovirus. In some embodiments, the virus itself may be is capable of lysosome disruption. Therefore, the nucleic acid protection component and the lysosome evading component are both components of the virus attached to the bead particle. Alternatively, the virus may not be capable of lysosome disruption. In such a case, a separate lysosome evading component may be added. Preferred viruses include HIV, adenovirus, Sindbis virus, and hybrid and recombinant versions thereof, such as an HIV-adenovirus hybrid, which is essentially a recombinant adenovirus that has been engineered to express HIV antigens. Viruses can be attached to the beads directly, using conventional methods, or indirectly. See Hammond et al., Virology 254:37-49 (1999).

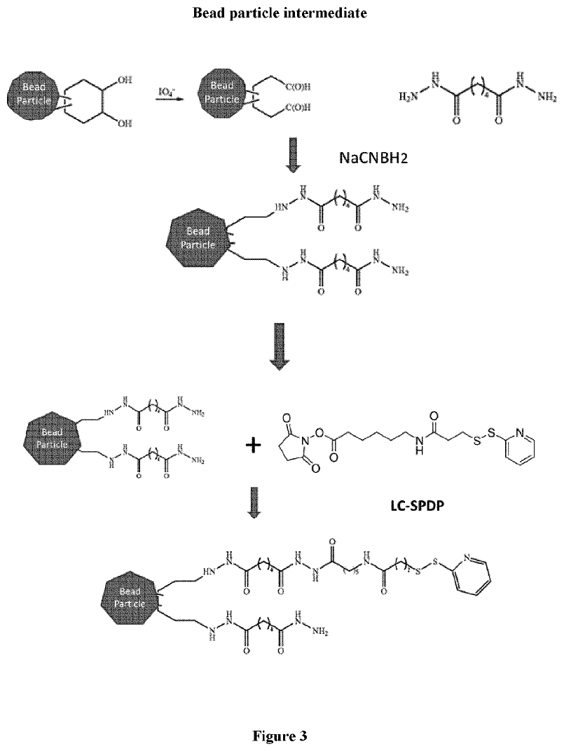

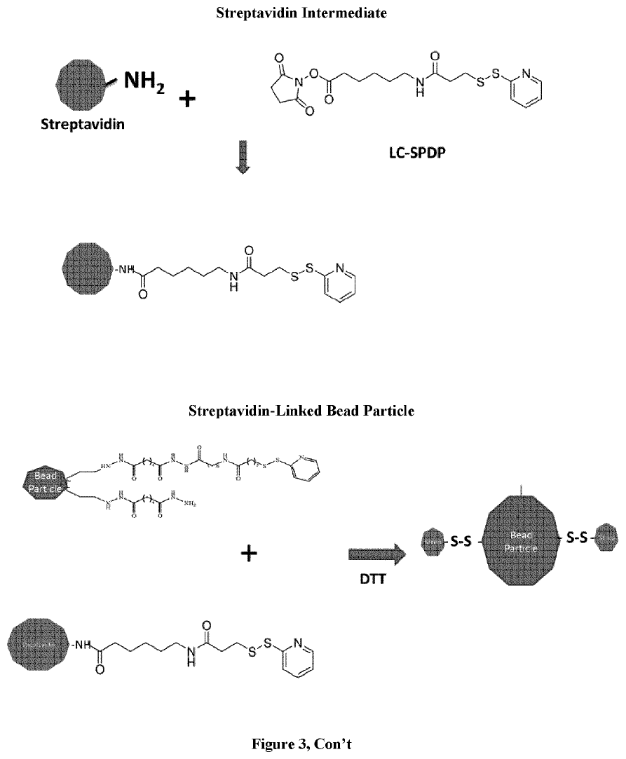

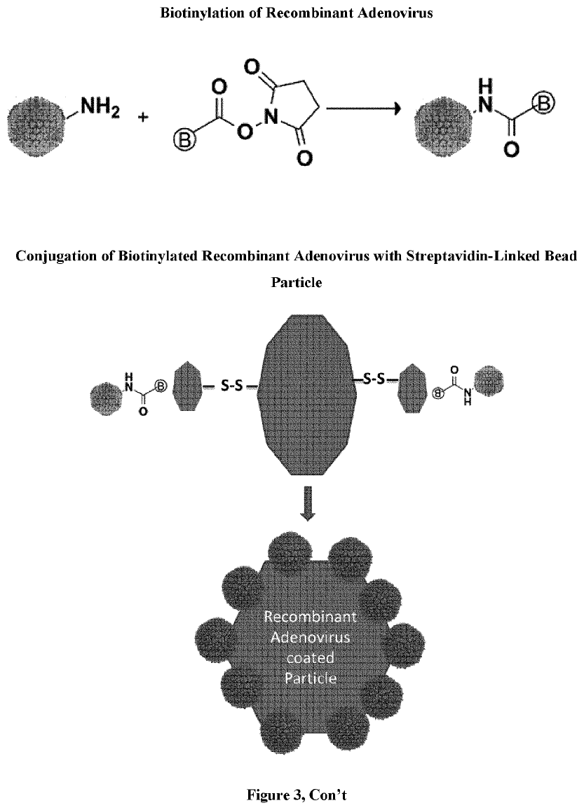

For example, in some embodiments, the target nucleic acids may be delivered in a recombinant adenovirus that is conjugated to a bead vector via a biotin-streptavidin linkage. The bead particle may be modified to attach a linker comprising streptavidin and the recombinant virus may be biotinylated as shown in FIG. 3.

Because viral infection is not essential for the nucleotide component of the disclosed bead vectors to reach the cytoplasm of the monocyte cell, the virus can also be replication/infection deficient. For example, one method for producing a replication/infection deficient adenovirus can be achieved by altering the virus fiber protein. Thus, in some embodiments, a virus in which the fiber protein is engineered by specific mutations to allow the fiber protein to bind to an antibody but not to its cognate cellular receptor can be used in the particles of the present disclosure.

Another method for producing a replication/infection deficient virus is by intentionally causing denaturation of the viral component responsible for infectivity. In the case of adenovirus, for example, the fiber protein could be disrupted during the preparation of the virus. For HIV, this could include the envelope (env) protein. Thus, in some embodiments, a method for creating an infection deficient virus for attachment to the disclosed bead particles comprises removing the outer membranes of the virus so that only the virus core remains. If a replication/infection deficient virus prepared as described above is attached to a bead particle, then a nucleic acid protecting component, as described above, may also be attached to the particle.

In some embodiments, it may be beneficial for the vector to stably integrate into the target cell chromosome. For example, one mode for achieving stable integration is through the use of an adenovirus hybrid. Such an adenovirus hybrid may comprise, for example, an adenoviral vector carrying retrovirus 5′ and 3′ long terminal repeat (LTR) sequences flanking the DNA component encoding a therapeutic nucleic acid or protein (e.g., a PD-1 ectodomain or an anti-CTLA4 antibody) and a retrovirus integrase gene (see Zheng, et al. Nature Biotechnology, 18:176-180, 2000).

In some embodiments, transient expression may be preferred and cytoplasmic viruses, like Sindbis virus, for example, can therefore be employed.

In some embodiments, where no lysosome evading component is naturally present on the virus, one may be added. For example, in the case of Sindbis or other such viruses, the virus can be engineered to express all or part of the adenovirus penton protein for the purpose of evading the lysosome.

### Methods for Attaching the Components to the Particle

Attachment of the components discussed above to the bead vector particle may be accomplished by any known means. As set out above, the various components can include a target nucleic acid (e.g., a PD-1 ectodomain or an anti-CTLA4 antibody), a lysosome evading component, which may both be present in a virus, and a nucleic acid protecting component. Although numerous methods of attaching such components are known in the art, specific methods for attachment may include, but are not limited to, antibody attachment, biotin-avidin/streptavidin interaction, and chemical crosslinking. Bead particles may be prepared with chemically attached antibodies, avidin, biotin, or other selective attachment sites.

Antibody attachment can occur via any antibody interaction. Antibodies include, but are not limited to, polyclonal antibodies, monoclonal antibodies (mAbs), humanized or chimeric antibodies, single chain antibodies including single chain Fv (scFv) fragments, Fab fragments, F(ab′)2 fragments, fragments produced by a Fab expression library, anti-idiotypic (anti-Id) antibodies, epitope-binding fragments, single-domain antibodies, and humanized forms of any of the above.

In general, techniques for preparing polyclonal and monoclonal antibodies as well as hybridomas capable of producing the desired antibody are well known in the art (Campbell, A. M., Monoclonal Antibody Technology: Laboratory Techniques in Biochemistry and Molecular Biology, Elsevier Science Publishers, Amsterdam, The Netherlands (1984); St. Groth et al., J. Immunol. Methods 35:1-21 (1980); Kohler and Milstein, Nature 256:495-497 (1975)), the trioma technique, the human B-cell hybridoma technique (Kozbor et al., Immunology Today 4:72 (1983); Cole et al., in Monoclonal Antibodies and Cancer Therapy, Alan R. Liss, Inc. (1985), pp. 77-96).

One example of antibody attachment encompassed may comprise a single antibody that is chemically affixed to the bead vector particle. The antibody is specific to the component to be attached to the particle.

Alternatively, two or more antibodies can be used. In this case, one antibody, attached to the bead, may be specific for a second antibody. The second antibody is specific to the component to be attached to the bead. Thus, the component-specific antibody binds the component, and that antibody, in turn, is bound by the bead-bound antibody. For instance, a goat- or rabbit-anti-mouse antibody may be bound to the bead and a mouse monoclonal antibody used to bind the specific component. Or, in another alternative format, the two or more antibodies my each be specific for a different component to be attached to the particle, such that the particle is decorated with two or more distinct components (i.e., two distinct viral particles, two distinct proteins, etc.)

In another example of antibody attachment, protein A, or any similar molecule with an affinity for antibodies, is employed. In this example, the beads are coated with protein A, which binds to an antibody, and, in turn is bound to the component being attached to the bead.

Attachment via biotin-avidin/streptavidin interaction may be accomplished, for instance, by attaching avidin/streptavidin to a bead vector particle and attaching biotin to the component to be attached. Chemical crosslinking may be accomplished by conventional means known to the artisan. See, e.g., FIG. 3.

Another attachment mechanism may comprise a nucleic acid serving as a multiple binding vehicle. Synthetic “gripper” protein nucleic acid (PNA) oligonucleotides are designed to specifically bind to different nucleic acid sequences. PNA is a polynucleic acid analog with a peptide backbone rather than a deoxyribosephosphate backbone. PNAs can be attached directly to the bead or derivatized for convenient attachment, thereby providing a sequence-specific means of attaching nucleic acid. Each gripper oligonucleotide can be derivatized or attached to different ligands or molecules and designed to bind different nucleic acid sequences. It is believed that the PNA interacts with the DNA via Hoogsteen base pairing interactions and that a stable PNA-DNA-PNA triplex clamp is formed (Zelphati, et al. BioTechniques, 28:304-316, 2000).

Thus, in one embodiment, one gripper is employed to bind the nucleic acid component to the bead and another is used to bind the lysosome evading component to the nucleic acid component. Many such iterations are possible. For example, a “gripper” comprising biotin can be sequence specifically bound at one site to the nucleic acid. Attachment to a particle coated with avidin occurs via biotin-avidin interaction. At another site on the nucleic acid, another “gripper” with a lysosome/phagasome evading component can be sequence specifically bound. Optionally, a “gripper” with a DNA protecting component can be sequence specifically bound to the nucleic acid at yet another site. Exemplary gripper oligonucleotides have been previously described.

In the case of attaching viruses to the bead particle, this can also be accomplished by engineering the virus to express certain proteins on its surface. For instance, the HIV env protein might be replaced with the adenovirus penton protein, or a portion thereof. The recombinant virus then could be attached via an anti-penton antibody, with attachment to the bead mediated, for example, by another antibody or protein A. In some embodiments, the penton protein also would serve as a lysosome evading component.

### Formulations

Pharmaceutical compositions suitable for use in the methods described herein can include the disclosed bead vectors and a pharmaceutically acceptable carrier or diluent.

The composition may be formulated for intravenous, intratumoral subcutaneous, intraperitoneal, intramuscular, oral, nasal, pulmonary, ocular, vaginal, or rectal administration. In some embodiments, the disclosed bead vectors are formulated for intravenous, subcutaneous, intraperitoneal, or intramuscular administration, such as in a solution, suspension, emulsion, etc. In some embodiments, the disclosed bead vectors are formulated for oral administration, such as in a tablet, capsule, powder, granules, or liquid suitable for oral administration. The pharmaceutical compositions can be formulated to be an immediate-release composition, sustained-release composition, delayed-release composition, etc., using techniques known in the art.

In some embodiments, the disclosed bead vectors may be formulated for parenteral administration by, for example, intravenous, intramuscular or subcutaneous injection. Formulations for injection may be presented in unit dosage form, e.g., in ampules or in multi-dose containers, optionally with an added preservative. The compositions may take such forms as suspensions, solutions or emulsions in oily or aqueous vehicles, and may contain formulatory agents such as suspending, stabilizing and/or dispersing agents. The bead vector may also be formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing.

In some embodiments, the bead vectors may be formulated to be administered concurrently with another therapeutic agent. In some embodiments, the bead vectors may be formulated to be administered in sequence with another therapeutic agents. For example, the bead vectors may be administered either before or after the subject has received a treatment of chemotherapy.

### Methods of Treatment

Provided herein are methods of treating tumors, cancer, malignant disease, or cancer cell proliferation with the disclosed bead vectors. More specifically, the disclosure provides for methods of inhibiting the PD-1 checkpoint in tumor/cancer cells through the expression of an extracellular domain of PD-1 or a PD-L1 specific antibody or binding fragment thereof. Similarly, provided herein are methods of inhibiting the CTLA4 checkpoint in tumor/cancer cells through the expression of an anti-CTLA4 antibody or binding fragment thereof, including a single-domain antibody. In some embodiments, the disclosed bead vectors may be used to drive expression of other checkpoint inhibitors, such as antibodies or fragments specific for the protein targets shown in Table 1. Such methods may comprise administering a therapeutically effective amount of the disclosed bead vectors containing a nucleic acid component for expression of a PD-1 extracellular domain, an anti-CTLA4 antibody, or an antibody or binding fragment that is specific for a checkpoint protein.

The disclosed bead vectors are highly selective for monocyte cells (e.g., macrophages or TAMs). It is, therefore, useful for any application involving selectively introducing a nucleic acid component into a monocyte cell. In some embodiments, the disclosed vectors are administered to treat cancer, and, in particular, solid tumors. Typical methods comprise contacting a monocytic cell with a bead vector. The administration of the bead vectors is not particularly limited due to the way the disclosed bead vectors function.

The bead vectors may be injected directly into a tumor or they may be administered, intradermally, subcutaneously, or systemically (i.e., into the peritoneal of the subject). There is a constant influx of macrophages into solid tumors, and therefore even marcophages that phagocytose the bead particles systemically may still infiltrate the tumor bed and function to treat the tumor or prevent tumor growth. Moreover, in some embodiments, the nucleic acid component of the bead vector may be under the control of a hypoxia-induced promoter, in which case the target gene or genes (i.e., a PD-1 extracellular domain, an anti-CTLA4 antibody, or an anti-checkpoint protein antibody) will only be expressed once the monocytic cell that phagocytosed it has infiltrated the tumor bed.

Alternatively or additionally, the bead vectors may function once phagocytosed by macrophages by being expressed in a lymph node in proximity to the tumor or cancer that is to be treated. In these embodiments, the disclosed bead vector may be administered intradermally or subcutaneously in an area proximate to the closest lymph node (i.e., the “target lymph notde”) to the tumor that is targeted for treatment. In this sense, administration proximate to the target lymph node means into or as close to the target lymph node as possible, but at least closer to the target lymph node than any other lymph node. Once in the target lymph node, the macrophages that phagocytosed the bead vectors will express the nucleic acid to produce a PD-1 extracellular domain, an anti-CTLA4 antibody, or an anti-checkpoint protein antibody that will travel to the tumor site and inhibit the targeted checkpoint within the tumor microenvironment.

This unique mechanism of action of the disclosed bead vectors is a dramatic improvement over the current state of checkpoint inhibiting therapeutics. Currently, checkpoint inhibitors such as Pembrolizumab (Keytruda), Nivolumab (Opdivo), Atezolizumab (Tecentriq), and Ipilimumab (Yervoy) are effective at treating various types of cancer. However, these drugs are administered systemically, and therefore, cause off-target effects that can be life threatening. Indeed, checkpoint inhibitors are known to cause a unique spectrum of side effects termed immune-related adverse events (irAEs), which can include dermatologic, gastrointestinal, hepatic, endocrine, and other organ system effects. The disclosed bead vectors prevent or minimize these off-target effects by expressing the encoded checkpoint inhibitor only within or nearby the tumor microenvironment, thus decreasing side effects through tumor targeting. As noted above, due to the constant infiltration of new macrophages into the tumor bed, the disclosed bead vectors may produce these improved effects when they are administered systemically (e.g., parenterally), intradermally, subcutaneously, or by directly injecting them into the tumor or target lymph node.

The bead vector can be contacted with monocyte cells either in vivo or in vitro. Hence, both in vivo and ex vivo methods are contemplated herein.

In some embodiments, in vivo methods comprise administering a bead vector pareterally, for example, intravenously, intramuscularly, subcutaneously or intradermally. In some embodiments, the bead vector is injected directly into the tumor. In some embodiments, a bead vector may be administered by bolus injection or continuous infusion.

In some embodiments, ex vivo methods comprise contacting monocytic cells outside the body and then administering the contacted cells to a patient in need thereof. The cells may also be administered parenterally, for instance, via infusion. Monocytic cells that are contacted by bead vectors in ex vivo methods may be autologous or allogeneic. Monocytic cells for use in ex vivo methods may be isolated by known methods of leukapheresis from a donor or from the patient (i.e., the ultimate recipient of the monocytic cells to be contacted with the disclosed bead vectors).

The disclosed bead vectors may be injected directly into the patient, yet are still able to deliver the target gene or genes (i.e., a PD-1 ectodomain, an anti-CTLA4 antibody, or an anti-checkpoint protein antibody) to the body's monocytic cells for the purpose of treating cancer, such as TAMs. Prior methods that targeted these cells rely principally on isolated a patient's monocytic cells and manipulating them in vitro and then returning the cells to the patient. While such embodiments are contemplated in the present disclosure, the disclosed bead vectors provide a substantial improvement because they may be used in both in vivo and ex vivo methods. Moreover, altering the route of administration can alter the monocytic cells targeted. For example, in the case of intravenous injection, macrophages may be targeted, and in the case of subcutaneous injection, dendritic cells may be targeted.

Thus, in some embodiments, targeting gene expression of a PD-1 ectodomain or an anti-CTLA4 antibody to a monocytic cell lineage using the disclosed bead vectors is effective for cancer treatment. One type of cancer treatment encompassed by the disclosure involves targeting expression of a PD-1 ectodomain or an anti-CTLA4 antibody to a solid tumor. In some embodiments, targeting gene expression of an anti-checkpoint protein antibody (i.e., an anti-PD-L1 antibody or an anti-CTLA4 antibody) to a monocytic cell lineage using the disclosed bead vectors is effective for cancer treatment.

It is known that as tumors (both primary tumors and metastases alike) grow beyond a few millimeters in diameter and become deficient in oxygen, creating a hypoxic microenvironment within the tumor. When such tumors become oxygen starved, they secrete signal proteins, such as angiogenic factors to increase the blood supply into the hypoxic areas of the tumor.

As a part of the mechanism of angiogenic induction, hypoxic tumors secrete a signaling chemokine protein that attracts monocytes to the tumor. Monocytes attracted to the sites of growing tumors then become macrophages and assist in the induction of tumor angiogenesis. Therefore, an effective method of tumor targeting involves administering a therapeutically effective amount of a bead vector containing a PD-1 ectodomain sequence, an anti-CTLA4 antibody, or an anti-checkpoint protein antibody sequence to a cancer patient, either directly or via ex vivo contact with monocytic cells. The monocyte cells containing the phagocytized bead vector are attracted to the tumor site and will selectively express the PD-1 ectodomain, an anti-CTLA4 antibody, or the anti-checkpoint protein antibody sequence in the tumor microenvironment.

In some embodiments, the target gene may encode a PD-1 ectodomain or extracellular domain. The PD-1 sequence may be derived from human, murine, rat, bovine, or other mammalian forms of PD-1. The PD-1 sequence may comprise a signaling domain, an extracellular domain, or a combination thereof. In some embodiments, the target gene may encode an anti-checkpoint protein antibody or binding fragment, such as an antibody or binding fragment that specifically binds one of the proteins in Table 1.

In some embodiments, the tumor or cancer being treated may express PD-1 ligands, such as PD-L1 and PD-L2. Thus, in some embodiments, the administration of the disclosed bead vectors will result in the expression of a PD-1 extracellular or ectodomain protein by tumor-associated macrophages. The expression of such PD-1 extracellular or ectodomain proteins in the tumor microenvironment can sequester all PD-1 ligands in the tumor microenvironment and result in inhibition of the PD-1 checkpoint.

In some embodiments, the target gene may encode an anti-CTLA4 antibody (e.g., a single-domain antibody). The anti-CTLA4 antibody may be derived from any known anti-CTLA4 antibody, for example, the anti-CTLA4 antibody disclosed in U.S. 2011/0044953.

In some embodiments, the tumor or cancer being treated may express CTLA4 or CTLA4 ligands, such as CD80 and/or CD86. Thus, in some embodiments, the administration of the disclosed bead vectors will result in the expression of an anti-CTLA4 and/or anti-CTLA4 ligand (e.g., CD80 or CD86) antibody by tumor-associated macrophages. The expression of such an antibody (e.g., a single-domain antibody) in the tumor microenvironment can sequester all CTLA4 or CTLA4 ligands in the tumor microenvironment and result in inhibition of the CTLA4 checkpoint.

In some embodiments, administration of the bead vectors will result in expression of an anti-checkpoint protein antibody by tumor-associated macrophages. For example, the TAMs that have phagocytosed the bead vectors will express an anti-PD-L1 and/or an anti-CTLA4 antibody in the tumor microenvironment, resulting in the inhibition of the PD-1 and/or CTLA4 checkpoint.

In some embodiments, the tumor or cancer being treated includes, but is not limited to, a neurological cancer, breast cancer, a gastrointestinal cancer (e.g., colon cancer), renal cell carcinoma (e.g., clear cell renal cell carcinoma), or a genitourinary cancer (e.g., ovarian cancer). In some embodiments, the cancer is melanoma, lung cancer (e.g., non-small cell lung cancer), head and neck cancer, liver cancer, pancreatic cancer, bone cancer, prostate cancer, bladder cancer, or a vascular cancer. Indeed, the disclosed methods provide a broad spectrum approach to treating tumors, cancer, malignant disease, or cancer cell proliferation, so the type of disease to be treated is not particularly limited.

Dosage regimens can be adjusted to provide the optimum desired response (e.g., a therapeutic response like tumor regression or remission). For example, in some embodiments, a single bolus of bead vectors may be administered, while in some embodiments, several divided doses may be administered over time or the dose may be proportionally reduced or increased as indicated by the situation. For example, in some embodiments the disclosed bead vectors may be administered once or twice weekly by subcutaneous or intravenous injection. In some embodiments, the disclosed bead vectors may be administered once or twice monthly by subcutaneous injection. In some embodiments, the disclosed bead vectors may be administered once every week, once every other week, once every three weeks, once every four weeks, once every other month, once every three months, once every four months, once every five months, or once every six months.

Furthermore, the disclosed methods of treatment can additionally comprise the administration of a second therapeutic compound in addition to disclosed bead vectors. For example, in some embodiments, the additional therapeutic compound may be a CAR-T cell, a tumor-targeting antibody, an immune response potentiating modality, a checkpoint inhibitor, or a small molecule drug, such as a BTK inhibitor (e.g. ibrutinib), an EGFR inhibitor (e.g. CK-101), a BET inhibitor (e.g. CK-103), a PARP inhibitor (e.g. olaparib or CK-102), a PI3Kdelta inhibitor (e.g. TGR-1202), a BRAF inhibitor (e.g. Vemurafenib), or other chemotherapeutics known in the art.

Particular treatment regimens may be evaluated according to whether they will improve a given patient's outcome, meaning the treatment will reduce the risk of recurrence or increase the likelihood of progression-free survival of the given cancer or tumor.

Thus, for the purposes of this disclosure, a subject is treated if one or more beneficial or desired results, including desirable clinical results, are obtained. For example, beneficial or desired clinical results include, but are not limited to, one or more of the following: decreasing one or more symptoms resulting from the disease, increasing the quality of life of those suffering from the disease, decreasing the dose of other medications required to treat the disease, delaying the progression of the disease, and/or prolonging survival of individuals.

Furthermore, while the subject of the methods is generally a cancer patient, the age of the patient is not limited. The disclosed methods are useful for treating tumors, cancer, malignant disease, or cancer cell proliferation with various recurrence and prognostic outcomes across all age groups and cohorts. Thus, in some embodiments, the subject may be a pediatric subject, while in other embodiments, the subject may be an adult subject.

The following examples are given to illustrate the present disclosure. It should be understood that the invention is not to be limited to the specific conditions or details described in these examples.

### EXAMPLES

### Example 1—Creation of Recombinant Virus for Expressing PD-1 Extracellular Domains

At least 5 μg of purified plasmid DNA of pAd-DEST expression construct was digested with Pac I restriction enzyme, and the digested plasmid DNA was gel purified.

The purified plasmid was resuspended in TE Buffer, pH 8.0 to a final concentration of 0.1-3.0 μg/μl. Roughly 5×10⁵ AD293 cells were plated into one well of a 6-well plate and cultured overnight.

Using lipofactamine 2000, about 1 μg of pacI digested plasmid DNA was transfected into AD293 cells. The culture medium was replaced with fresh, complete culture medium every 2-3 days until visible regions of cytopathic effect (CPE) were observed (typically 7-10 days post-transfection). Adenovirus-containing cells were harvested at approximately 80% CPE by squirting cells off the plate with a 10 ml tissue culture pipette. The cells and media were transferred to a sterile, 15 ml, capped tube.

A Crude Viral Lysate was prepared by performing at least three freeze-thaw cycles. The lysate was centrifuged at 3000 rpm for 15 minutes at room temperature to pellet the cell debris. The supernatant containing viral particles was transferred to cryovials in 1 ml aliquots and stored as viral stocks at −80° C. This stock was used for viral amplification.

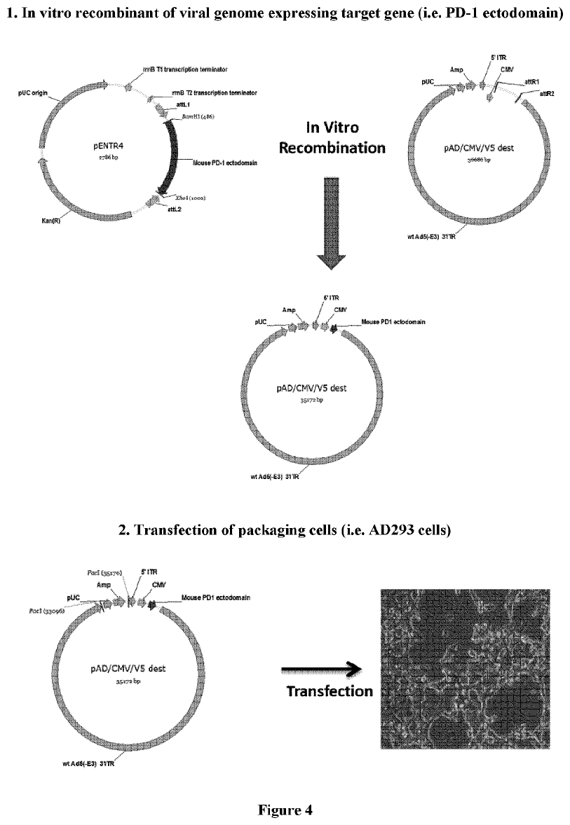

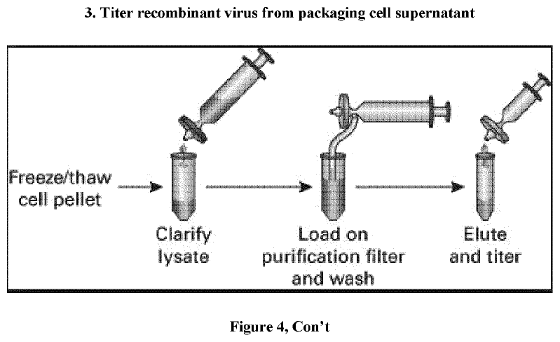

FIG. 4 shows an exemplary scheme for making recombinant viruses according to this method. Such recombinant viruses can be attached to bead particles of the disclosure using any means, for example, by biotin/streptavidin conjugation as shown in FIG. 3.

### Example 2—Treatment of Mice Bearing Melanoma Tumors

C57 B6 mice were injected with 1×10⁶ B16 murine melanoma cells. After twelve (12) days, the mice had palpable xenograft tumors.

Twelve days after the injection of the B16 murine melanoma cells, mice were treated with one of two bead particles. Control mice received a direct intratumor injection of 1×10⁶ bead particles containing 1×10⁷ green fluorescence protein (GFP)-expressing adenovirus. Mice in the experimental group received a direct intratumor injection of 1×10⁶ bead particles containing 1×10⁷ PD-1 adenovirus designed to express the extracellular domain of mouse PD-1.

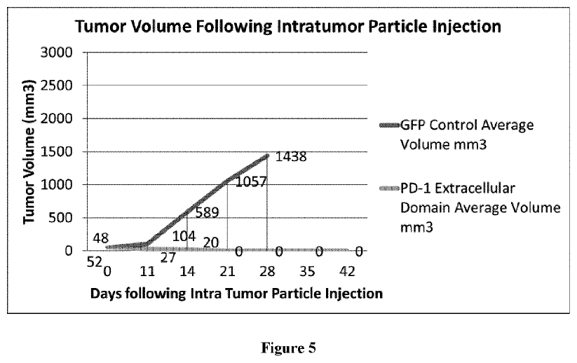

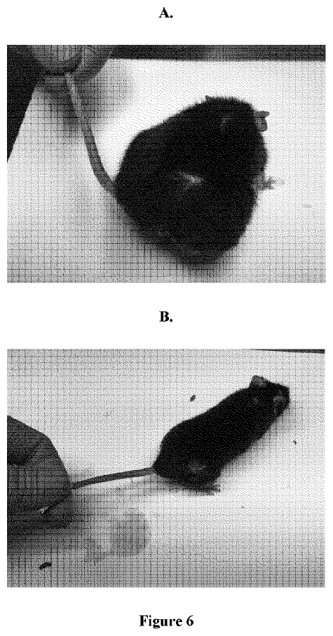

The volume of each mouse's tumor was measured following the intra-tumor injection. Tumor volumes are shown in FIG. 5. All mice in the control group died on or before day 28 post intra-tumor injection. All mice in the experimental group that received bead particles for expressing a PD-1 extracellular domain survived beyond day 45 post intra-tumor injection, and their tumor volumes decreased.

FIG. 6 shows (A) a control mouse and (B) a mouse that received bead particles for expressing a PD-1 extracellular domain four weeks after a single dose treatment of control or experimental particles, respectively. As can be seen in the figure, the control mouse has a large tumor, while the mouse from the treatment group has little or no visible tumor growth.

### Example 3—Prophetic Human Treatment

This example illustrates methods using the disclose bead vectors in the treatment of cancer.

A patient known to have or suspected of having cancer is administered a therapeutically effective amount of a bead vector comprising a nucleic acid encoding a PD-1 extracellular domain or an anti-CTLA4 antibody (e.g., a single-domain antibody), by intravenous, intradermal, or subcutaneous injection. The patient is evaluated for the presence and/or severity of signs and symptoms associated with cancer, including, but not limited to, pain, weakness, tumor size, etc., and the patient is treated until one or more signs/symptoms are reduced, ameliorated, or eliminated. Optionally, samples may be taken from the patient to monitor cancer progression following treatment. Optionally, another dose of the bead vector comprising a nucleic acid encoding a PD-1 extracellular domain or an anti-CTLA4 antibody is administered if signs/symptoms persist and/or if the cancer progresses or recurs.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

1. A bead vector comprising (i) a nucleic acid encoding an anti-CTLA4 antibody, (ii) a lysosome evading component, and (iii) a bead particle that can be phagocytized.

2. The bead vector of claim 1, wherein the lysosome evading component is a non-infectious virus or non-infectious component of a virus.

3. The bead vector of claim 2, wherein the non-infectious virus is an adenovirus.

4. The bead vector of claim 3, wherein the adenovirus is a recombinant adenovirus.

5. The bead vector of claim 2, wherein the non-infectious virus or non-infectious component of a virus is non-replicative.

6.-11. (canceled)

12. The bead vector of claim 1, wherein the nucleic acid encoding an anti-CTLA4 antibody is encoded in an expression vector comprising the sequence shown in FIG. 8.

13. The bead vector of claim 1, wherein the anti-CTLA4 antibody is a single-domain antibody.

14. (canceled)

15. The bead vector of claim 1 further comprising a nucleic acid protecting component selected from the group consisting of protamine, polyarginine, polylysine, histone, histone-like proteins, synthetic polycationic polymers and a core particle of a retrovirus with the appropriate packaging sequence included in the nucleic acid sequence.

16. (canceled)

17. (canceled)

18. The bead vector of claim 1, wherein the lysosome evading component comprises an adenovirus penton protein.

19. (canceled)

20. (canceled)

21. The bead vector of claim 1, wherein the bead particle comprises a yeast cell wall particle (YCWP).

22. A bead vector for directed entry into a monocyte cell comprising (i) a nucleic acid encoding an anti-CTLA4 antibody, (ii) a lysosome evading component, and (iii) a bead particle that is about 0.5 to about 2.5 microns and allows the composition to be phagocytized by a monocytic cell.

23. The bead vector of claim 22, wherein the monocytic cell is a macrophage.

24. The bead vector of claim 23, wherein the macrophage is a tumor-associated macrophage (TAM).

25.-32. (canceled)

33. A method of treating cancer in a patient comprising administering to a patient with cancer a bead vector comprising (i) a nucleic acid encoding an anti-CTLA4 antibody, (ii) a lysosome evading component, and (iii) a bead particle that is about 0.5 to about 2.5 microns, wherein administration of the bead vector treats the patient's cancer.

34.-47. (canceled)

48. A bead vector comprising (i) a nucleic acid encoding a PD-1 extracellular domain (ii) a lysosome evading component and (iii) a bead particle that can be phagocytized.

49.-69. (canceled)

70. A bead vector for directed entry into a monocyte cell comprising (i) a nucleic acid encoding a PD-1 extracellular domain (ii) a lysosome evading component and (iii) a bead particle that is about 0.5 to about 2.5 microns and allows the composition to be phagocytized by a monocytic cell.

71.-83. (canceled)

84. A method of treating cancer in a patient comprising administering to a patient with cancer a bead vector comprising (i) a nucleic acid encoding a PD-1 extracellular domain (ii) a lysosome evading component and (iii) a bead particle that is about 0.5 to about 2.5 microns, wherein administration of the bead vector treats the patient's cancer.

85.-98. (canceled)

99. A bead vector comprising (i) a nucleic acid encoding an anti-checkpoint protein antibody or binding fragment thereof (ii) a lysosome evading component and (iii) a bead particle that can be phagocytized.

100.-113. (canceled)

114. A bead vector for directed entry into a monocyte cell comprising (i) a nucleic acid encoding an anti-checkpoint protein antibody or binding fragment thereof (ii) a lysosome evading component and (iii) a bead particle that is about 0.5 to about 2.5 microns and allows the composition to be phagocytized by a monocytic cell.

115.-125. (canceled)

126. A method of treating cancer in a patient comprising administering to a patient with cancer a bead vector comprising (i) a nucleic acid encoding an anti-checkpoint protein antibody or binding fragment thereof (ii) a lysosome evading component and (iii) a bead particle that is about 0.5 to about 2.5 microns, wherein administration of the bead vector treats the patient's cancer.

127.-138. (canceled)


SEQUENCE LISTING (4 sequences)

SEQ ID NO: 1 - protein, 6 aa, Mus sp.
VAYEEL

SEQ ID NO: 2 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - protein, 288 aa, Mus sp.
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 4 - DNA, 13210 bp, Artificial Sequence
  Description of Artificial Sequence Synthetic polynucleotide
taatacgactcactatagggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatacatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccatgaattacatccctacgcaaacgttttacggccgccggtggcgcccgcgcccggcggcccgtccctggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaagggtagggtagtcgctatcgtcctgggcggggccaacgagggctcacgcacagcactgtcggtggtcacctggaacaaagatatggtgactagagtgacccccgaggggtccgaagagtgggatctatggagacagacacactcctgctatgggtactgctgctctgggttccaggttccactggtgacgatatcaggcgcgccgacattgtgatgacccagactacactttccctgcctgtcagtcttggagatcaagcctccatctcttgcagatctagtcagagcattgtacatagtaatggaaacacctatttaggatggtacctgcagaaaccaggccagtctccaaagctcctgatctacaaagtttccaaccgattttctggggtcccagacaggttcagtggcactggatcagggacagatttcacactcaagatcagcagagtggaggctgaggatctgggagtttattactgctttcaaggttcacatgttccttacacgttcggaggggggaccaagctggaaataaaacgggctgatgctgcaccaactgtatccggatccggaggtgggagtggtggcggaagtggcggagggagcgaggcaaagctgcaggagtctggacctgtgctggtgaagcctggggcttcagtgaagatgtcctgtaaggcttctggatacacattcactgactactatatgaacttggtgaagcaaagccatggaaagagccttgagtggattggagttattaatccttataacggtgatactagctacaaccagaagttcaagggcaaggccacattgactgttgacaagtcctccagcacagcctacatggagctcaacagcctgacatctgaggactctgcagtctattactgtgcaagatactatggttcctggtttgcttactggggccaagggactctgatcactgtctctacagccaaaacaacacccccatcagtctatccactggcccctagatcttctcgagaacaaaaactcatctcagaagaggatctgcatcatcatcaccatcactaacgaatcgatgcatcctagggcccgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccg